(19) United States
(12) Patent Application Publication
(10) Pub. No.: US 2009/0247425 A1
(43) Pub. Date: Oct. 1, 2009

(54) COMPOSITIONS AND METHODS FOR REUSING ARRAYS

(75) Inventors: Hua Zhang, Carlsbad, CA (US); Mark Andersen, Carlsbad, CA (US)

Correspondence Address: LIFE TECHNOLOGIES CORPORATION;C/O INTELLEVATE, P.O. BOX 52050, MINNEAPOLIS, MN, 55402, US

(73) Assignee: INVITROGEN CORPORATION, Carlsbad, CA (US)

(21) Appl. No.: 12/058,531

(22) Filed: Mar. 28, 2008

(51) Int. Cl. C40B 40/06; C40B 50/00; C12N 9/24; C12N 9/12; C12N 9/16; C40B 50/14

(52) U.S. Cl. 506/16; 506/23; 506/30; 435/200; 435/196; 435/194

(57) ABSTRACT

The present disclosure relates generally to compositions and methods for the reuse of arrays, including microarrays. Specifically, the present disclosure discloses polynucleotide targets comprising nucleotide analogs that are not present within the probe polynucleotides immobilized on the array. The nucleotide-analog containing targets can be chemically modified to reduce their thermal stability and thus easier to remove from the array. In preferred embodiments, the disclosure relates to DNA probes hybridized to single-stranded deoxyribouridine-containing targets, the targets subsequently being chemically modified using a uracil DNA glycosylase and/or nuclease. Accordingly, the disclosure allows for the glycosylase treated, deoxyuridine-containing targets to be removed from the array by exposure to less stringent denaturing conditions than otherwise would have been required. Using less stringent denaturing conditions permits reuse of the array by reducing damage to the probe polynucleotides immobilized on the array during target removal.

### FIELD OF THE INVENTION

The present disclosure relates generally to nucleic acid arrays, and more specifically to the reuse of nucleic acid arrays.

### BACKGROUND INFORMATION

Array analysis generally uses pieces of DNA or RNA (“probes”) deposited onto known locations of a substrate (e.g., specially coated glass slides), followed by hybridization of labeled DNA or RNA molecules (“targets”) onto the array for subsequent analysis and comparison. For instance, DNA from a test sample and a reference sample can be labeled differentially using different fluorophores, and then hybridized to probes contained on the array. The ratio of the fluorescence intensity of the test sample to that of the reference sample is then calculated to measure relative differences in copy number for a particular location in the genome.

An important aspect of most, if not all, array studies is the replication of data. The availability of sample replicates is a significant advantage since the more replicates performed, the more statistical power gained. In many instances, a single DNA microarray is used only once. Therefore, the expense of performing replicate microarray experiments greatly adds to the overall cost, and in many cases, is impractical due to economic constraints.

Currently, several techniques allow labeled targets to be removed from an array, which can allow for reuse of the array and reduce lower costs. Such removal procedures may be referred to as “stripping” the array. Most “stripping” protocols, however, typically use relatively harsh treatments; that is, conditions more harsh than were used during the hybridization of the targets to the probes, and during the removal of any background signal. In some cases, scientists attempt to reuse arrays by removing targets with, for example, thermal or chemical means. For instance, arrays might be heated to temperatures around 95° C. to “melt off” the hybridized targets from the probes of the array. These temperatures are above the melting temperature (Tm) of hybridized nucleotide complexes, and therefore can allow the targets hybridized to the probes to disassociate from the array. Unfortunately, these temperatures are also high enough to cause damage to the probes themselves. Other stripping methods involve the use of formaldehyde and DMSO, which can also damage the probes of the array. Thus, even when these stripping treatments successfully remove the targets from the array, they can damage the probes on the array, thereby making the data from the damaged array unreliable, and also limiting the number of times an array can be reused. Consequently, there is a need in the field for a method of stripping arrays which minimally impacts the integrity of the array itself.

### SUMMARY OF THE INVENTION

The present disclosure provides compositions, methods and kits that allow for the reuse of arrays, particularly nucleic acid arrays, while minimizing damage to the arrays themselves. The result is that arrays that were previously considered not to be reusable (e.g., having relatively long targets hybridized thereto) now can be reused, and arrays that previously could only undergo a limited number of stripping procedures before a significant loss of quality was observed can now be reused a greater number of times with less decrease in quality after each subsequent stripping procedure.

In one aspect, the present disclosure provides methods for rendering a nucleic acid array suitable for repeated use. Some of these methods involve: (a) providing targets comprising a nucleotide analog that confers susceptibility to a degradative agent; (b) providing an array having probes attached thereto; (c) performing hybridization of the targets to the probes; and (d) incubating the array with the degradative agent, so that the targets are substantially removed from the array. The present disclosure also provides methods for removing targets hybridized to probes of a nucleic acid array, where the targets include a nucleotide analog that is susceptible to modification by a degradative agent. These methods also involve incubating the array with the degradative agent, thereby substantially removing the targets from the array.

In some embodiments of the disclosed methods, the degradative agent chemically modifies the nucleotide analogs (e.g., rendering the nucleotide analogs abasic or reducing the Tm of the hybridized target and probe). In some embodiments the chemically modified targets are removed from the immobilized probes through heat, protease, or nuclease (e.g., apurinic or apyrimidinic endonuclease) treatment, and/or dilution.

In some embodiments of the disclosed methods, the probes are immobilized on a solid support (e.g., by covalent bonding). In some embodiments the array is a microarray. In some embodiments the array is a spatially-addressable array. In some embodiments the array is a bead-based array. In some embodiments the probes are DNA.

In some embodiments of the disclosed methods, the targets are detectably labeled (e.g., with a fluorescent label or biotin). In some embodiments the targets are single-stranded. In some embodiments the targets are DNA.

In some embodiments of the disclosed methods, the nucleotide analog is deoxyuridine monophosphate (dUMP) or deoxyuridine triphosphate (dUTP). In some embodiments the degradative agent is a glycosylase (e.g., uracil DNA glycosylase).

Some embodiments of the disclosed methods involve targets having one or more nucleotide analogs not present in any or most of the probes immobilized on the array.

The disclosed methods may further involve hybridizing a different set of targets to the probes. The different set of targets may also include one more nucleotide analogs that are susceptible to the degradative agent.

In another aspect, the present disclosure provides compositions including a nucleic acid array where probes are hybridized to targets having nucleotide analog(s) that are susceptible to modification by a degradative agent. The compositions also include the degradative agent. The probes may be immobilized on a solid support, e.g., by covalent binding. The probes may be DNA. The array may be a microarray, and/or may be a spatially-addressable array. The targets may be detectably labeled, for example with a fluorescent or biotin label. The targets may be DNA. The targets may be single-stranded. The nucleotide analog may be deoxyuridine monophosphate (dUMP) or deoxyuridine triphosphate (dUTP). In some embodiments, the nucleotide analogs are not present in any or most of the probes immobilized on the array. The degradative agent can be a glycosylase, such as a uracil DNA glycosylase. The disclosed compositions may also include a nuclease or a stripping buffer.

In another aspect, the present disclosure provides kits for removing targets hybridized to an array. Some of the kits include (a) one or more stripping buffers; and (b) one or more glycosylases. The stripping buffer may include one or more of the following: at least 5% (w/v or v/v) formamide, at least 0.0001% (v/v or w/v) detergent, at least 0.01 nM monovalent cation salt, or at least 0.2 nM OH⁻. The kits may also include one or more nucleases (e.g., apurinic/apyrimidinic endonucleases), nucleotide analogs, and/or nucleic acid polymerases.

As used in the claims and specification, the words “comprising” (and any form of comprising, such as “comprise” and “comprises), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. The use of the word “a” or “an” when used in conjunction with the term “comprising” in the claims or specification may mean “one,” but it is also consistent with the meaning of “one or more,” “at least one,” and “one or more than one.” The term “substantially” and its variations are defined as being largely but not necessarily wholly that which is referenced as understood by one of ordinary skill in the art.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will be apparent to those skilled in the art from the claims and specification.

### BRIEF DESCRIPTION OF THE DRAWINGS

FIG. 1. DNA yield for synthesis of oligonucleotides incorporating dUTP versus dTTP.

FIG. 2. Results of assessment of labeling efficiency using dye incorporation with dTTP versus dUTP.

FIG. 3. Quantitative PCR assay showing dUTP incorporation into oligonucleotides.

FIG. 4. Stability assay for dTTP-containing and dUTP-containing oligonucleotides under denaturing conditions.

FIG. 5. Array images before and after UDG treatment.

FIG. 6. Percentage of average signal remaining after UDG treatment. T: dTTP sample. U: dUTP sample. G: Green channel (AF3). R: Red channel (AF5).

FIGS. 7A and 7B. The plot of green channel signal for array features before and after UDG treatment on array hybridized with (A) T-DNA and (B) U-DNA.

FIGS. 8A and 8B. The plot of red channel signal for array features before and after UDG treatment on array hybridized with (A) T-DNA and (B) U-DNA.

FIG. 9. Array images of a positive control and a rehybridized array following UDG treatment.

FIG. 10. Percentage of remaining signal versus original signal in the green channel of the b 100 brightest array features.

FIGS. 11A and 11B. The signal (A) and signal to noise (B) following three array rehybridizations.

### DETAILED DESCRIPTION OF THE INVENTION

Provided are compositions and methods for reusing nucleic acid arrays. The methods generally involve the removal of targets hybridized to probes of a nucleic acid array, where the targets comprise a nucleotide analog that is susceptible to modification by a degradative agent. The methods involve incubating the array with the degradative agent, thereby substantially removing the targets from the array. The provided methods can involve: (a) providing targets comprising a nucleotide analog that confers susceptibility to a degradative agent; (b) providing an array having probes attached thereto; (c) performing hybridization of the targets to the probes; (d) incubating the array with the degradative agent, so that the targets are substantially removed from the array.

Arrays

A nucleic acid array is a plurality of nucleic acids assembled as a collection of individually identifiable, soluble molecules (e.g., having a molecular bar code), or a collection of molecules fixed to beads, fibers, silica chips, slides, nanostructures, or other solid supports. The nucleic acid molecules (e.g., DNA, RNA, PNA, and conjugates thereof) fixed to or forming an array are referred to as “probes.” As used herein, the term array refers to an assembled collection of probes in its entirety.

An array can be an assembly composed of a support (also called a substrate) and nucleic acid molecules (or “probes”) immobilized on the support. The support may be, for example, a microscope glass slide, silicon chip or nylon membrane. An intentionally-created collection of probes may be immobilized to a solid support in a manner such that the identity of each probe at a given region is known or can be determined. In such embodiments, each probe that corresponds to a specific nucleic acid sequence is immobilized at a determined position on the support, which is sometimes referred to as a spot or “feature.”

Generally, the probes are immobilized on the support, for example by covalent bonding. They can be immobilized directly to the support, or in an indirect manner, for example, through the intermediate use of “linker” that are immobilized directly to the support. This can involve arrays for which the probes have been produced beforehand and then fixed on the support, or for which the probes are synthesized directly on the support. Any suitable method of producing an array can be used in the practice of the present disclosure.

One important type of array comprising a solid support is the microarray. Nucleic acid or DNA microarrays are miniaturized systems for genetic analysis on a large scale, enabling the study, for example, of the transcriptional activity of a large number of genes (genetic expression analysis), and the determination of the sequence of a large number of DNA fragments (including genetic polymorphism analyses), etc. Their general principle consists of fixing nucleic acid molecules on a support in an organized way, for example as a spatially-addressable array, and in a miniaturized manner so that it is possible to attach a large number of different nucleic acid molecules on a reduced surface. Microfabricated arrays of large numbers of probes are sometimes referred to as “DNA chips.” Many commercially available microarrays contain 96, 384, 1536, and higher quantity patterns of features.

An array is reusable if (1) it can be used to bind and thereby detect the presence or absence of one or more nucleic acid molecules in a sample, (2) targets used to detect the presence or absence of the nucleic acid molecules in a sample can be removed from the array, and (3) a second or subsequent sample can be assayed for the presence or absence of one more nucleic acid molecules using the same array. In certain embodiments of the present disclosure, all or substantially all of the targets are removed from the array prior to using the array to analyze a second or subsequent sample.

Nucleic Acid Targets

Nucleic acid molecules that hybridize to an array or microarray for analysis are referred to as “targets.” During hybridization, targets will attach themselves in a specific manner to the probes present on the array, whose nucleic sequence is similar to all or part of the sequence of the targets.

The term “nucleic acid molecules” refers to nucleic acids (e.g., DNA, RNA, PNA and conjugates thereof). The terms “nucleic acid,” “nucleic acid molecule,” oligonucleotide,” and “polynucleotide” may be used interchangeably, and refer to a sequence of two or more nucleotides, i.e., it is a deoxyribonucleotide or ribonucleotide polymer in either single-or double-stranded form, or multiple stranded form, or a combination or hybrid thereof. Nucleic acids include sequences of deoxyribonucleic acid (DNA) and/or ribonucleic acid (RNA), which may be isolated from natural sources, recombinantly produced, or artificially synthesized. A further example of a nucleic acid is peptide nucleic acid (PNA). In some nucleic acids there is nontraditional base pairing such as Hoogsteen base pairing, which has been identified in certain tRNA molecules and postulated to exist in a triple helix. With reference to polynucleotides, an “n-mer” is a single-stranded polynucleotide of “n” number of nucleotides.

“Nucleotides” or “nucleic acid bases” are compounds containing a nitrogenous heterocyclic base bound to a phosphorylated sugar by an N-glycosyl link. Nucleotides are the basic subunits of DNA and RNA. DNA nucleotides contain deoxyribose sugars; RNA nucleotides contain ribose sugars. The term includes, but is not limited to, the predominant naturally occurring nucleotides including the ribonucleoside triphosphates, such as adenosine triphosphate (ATP), cytidine triphosphate (CTP), guanosine triphosphate (GTP), thymidine triphosphate (TTP) or uridine triphosphate (UTP), and the deoxyribonucleotide triphosphates, such as deoxyadenosine triphosphate (dATP), deoxycytidine triphosphate (dCTP), deoxyguanosine triphosphate (dGTP), deoxythymidine triphosphate (dTTP) or deoxyuridine triphosphate (dUTP), as well as the corresponding ribonucleoside and deoxyribonucleoside diphosphates and monophosphates. A “nucleoside” is a base-sugar combination, i.e., a nucleotide lacking phosphate. It is recognized in the art that there is a certain inter-changeability in usage of the terms nucleoside and nucleotide. For example, the nucleotide deoxyuridine triphosphate, dUTP, is a deoxyribonucleoside triphosphate. After incorporation into DNA, nucleotides can be referred to, for example, as deoxyuridylate, deoxyuridine monophosphate (dUMP), deoxyuridine (dU)-containing DNA or dU-DNA.

A nucleic acid sequence is made up of four nucleic acid bases attached to a backbone. Generally, DNA is formed from a backbone comprised of 2′-deoxyribose subunits joined to one of four heterocyclic nitrogen-containing bases: adenine, guanine, cytosine or thymidine. These bases are capable of forming hydrogen bonds between complementary base pairs: A with T and C with G. DNA strands that are base paired in this manner are said to be “hybridized.” Similarly, RNA is formed from a backbone of ribose subunits joined to one of four hetercyclic nitrogen-containing bases: adenine, guanine, cytosine or uracil (instead of thymidine.) RNA's complementary base pairs are A with U and C with G. In some instances natural or artificial DNA or RNA nucleic acid sequences can also incorporate one or more base analogs which have identical, similar, or different base-pairing properties than A, T, U, C and G.

The terms “nucleotide” and “nucleic acid base” also includes nucleotide analogs. These analogs are those molecules having some structural features in common with a naturally occurring nucleotide such that they can be incorporated into a polynucleotide sequence. Preferably, nucleotide analogs do not prevent the hybridization of a nucleic acid into which they have been incorporated with a complementary polynucleotide in solution and/or on solid surface.

The term “nucleotide analog” as used herein refers to a nucleotide which is generally not naturally incorporated into human DNA (i.e. not ATP, GTP, CTP, TTP, dATP, dGTP, dCTP or dTTP, or diphosphates or monophosphates of the same) or into human RNA sequences (i.e. not ATP, GTP, CTP, UTP, dATP, dGTP, dCTP or dUTP, or diphosphates or monophosphates of the same). Nucleotide analogs are therefore those molecules having some structural features in common with a naturally occurring nucleotide such that when incorporated into a polynucleotide sequence, they allow hybridization with a complementary polynucleotide in solution and/or on solid surface.

A nucleotide analog may have a modified ribose moiety, a phosphodiester moiety, and/or a modified base moiety. Deoxyuridine is an example of a base-modified nucleotide analog with regard to DNA, but not RNA. Although the triphosphate form of deoxyuridine, dUTP, is present in living organisms as a metabolic intermediate, it is rarely incorporated into DNA. When dUTP is incorporated into DNA, the resulting deoxyuridine monophosphate (dUMP) is promptly removed in vivo by normal processes, e.g., processes involving the enzyme uracil DNA glycosylase (UDG). Thus, dUMP occurs rarely if ever in natural DNA. However, the uracil base found in dUMP is capable of hybridizing with a complementary dAMP nucleotide.

Examples of nucleotide analogs other than deoxyuridine nucleotides include those bases existing in nature such as hypoxanthine, xanthine, and inosine; those having alkylated and halogenated bases such as the N-methyl purines 5-methyladenine (5-meA), 7-methyladenine (7-meA), 3-methylguanine (3-meG) and 7-methylguanine (7-meG), 5-methylcytosine (5-meC), 7-hydroxyethylguanine, 7-chloroethylguanine, O2-alkylthymine, O2-alkylcytosine and 5-fluorouracil; those having oxidized and ring-fragmented bases including 8-oxoguanine, 7,8-dihydro-8-oxoguanine, 2,5,-amino-5-formamidopyrimidine (Fapy), 4,6-diamino-5-formamidopyrimidine (FapyAde), 2,6-diamino-4-hydroxy-5-formamidopyrimidine (FapyGua), 5-hydroxycytosine, 5,6-dihydrothymine, 5-hydroxy-5,6-dihydrothymine, thymine glycol, uracil glycol, isodialuric acid, alloxan, 5,6-dihyrouracil, 5′-hydroxy-5,6-dihydrouracil, 5-hydroxyuracil, 5-formyluracil, 5-hydroxymethyluracil, urea, methyltartronylurea and 5-hydroxyhydantoin; those having deaminated bases including deoxyinosine (hypoxanthine); and those having other bases including 1,N6-ethenoadenine, 3,N4-ethenocytosine and cyclobutane-pyrimidine dimer.

It should be noted here that throughout this disclosure various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, the description of a range such as 4 to 50 should be considered to have specifically disclosed all sub-ranges such as 4 to 10, 4 to 20, 4 to 30, 4 to 40, 4 to 50, 5 to 10, 5 to 20, etc., as well as individual numbers within that range, for example, 6, 8, 15, 20, 32, 39, 43, 48, etc. This applies regardless of the breadth of the range. Likewise, a description of a range such as 1 or more, 10 or more, etc. should be considered to have specifically disclosed individual numbers within that range as well as higher numbers, for example, 20, 50, etc.

Numerous methods of generating nucleic acid targets containing nucleotide analogs are known in the art and any suitable method can be used in the present invention. For example, DNA or cDNA targets containing nucleotide analogs can be generated using a DNA polymerase or reverse transcriptase, suitable primers, a suitable set of template nucleic acids (e.g., gDNA, mRNAs or other template polynucleotides), and an appropriate mixture of dNTPs comprising a deoxyribonucleotide triphosphate analog such as dUTP or d(5-meC)TP. Similarly, RNA targets containing nucleotide analogs can be generated using an RNA polymerase, suitable primers, a suitable set of template polynucleotides and an appropriate mixture of NTPs comprising a ribonucleotide triphosphate analog such as (5-meC)TP or (7-meA)TP. The ribonucleotide or deoxyribonucleotide triphosphate analog can make up any suitable percentage of the dNTPs or NTPs used in the target synthesis. Methods of determining a suitable quantity of dNTPs or NTPs in order to achieve a desired result are routine in the art, and will take into account the totality of the synthesis conditions, including the nature of the template, the nucleotide analog used, and the properties of enzyme used in synthesis with regard to the incorporation of different nucleotides and nucleotide analogs.

In general, the greater the percentage of nucleotide analogs present in the targets used, the greater the degree of degradation that can be achieved upon treatment with an agent that is capable of degrading the nucleotide analog(s) present in the target.

Hybridization and Detection of Hybridized Targets

The term “hybridization” as used herein refers to the process in which two or more single-stranded nucleic acid molecules bind non-covalently to form a multi-stranded (e.g., double stranded) nucleic acid molecule. The resulting multi-stranded nucleic acid molecule is a “hybrid.” Hybrids can contain, for example, two or more DNA strands, two or more RNA strands, or one or more DNA and one or more RNA strands.

The likelihood of two or more complementary polynucleotides hybridizing to form a stable complex (e.g., duplex), or once formed, the likelihood of the complex coming apart, depends on the thermal stability (Tₘ) of the complex formed between the polynucleotides. This is the temperature corresponding to the midpoint in the observed transition from double-stranded duplex to single-stranded form. One well-known factor affecting the Tₘ arises from the fact that adenine-thymine (A-T) duplexes have a lower Tₘ than guanine-cytosine (G-C) duplexes, due in part to the fact that A-T duplexes have 2 hydrogen bonds per base-pair, while G-C duplexes have 3 hydrogen bonds per base pair. Another significant factor is the overall length of the complex formed, where longer complexes have progressively higher Tₘ than shorter duplexes. Similarly, the degree to which the strands are complementary will also affect Tₘ. The greater the percentage of mismatches between the hybridized strands, the lower the Tₘ. Finally, the chemical environment in which the hybridization takes place can also significantly affect the Tₘ of the hybridized complex.

As used herein, “stringency” refers to the conditions of a hybridization reaction that influence the degree to which polynucleotides hybridize. Stringent conditions can be selected that allow polynucleotide duplexes to be distinguished based on their degree of mismatch. High stringency is correlated with a lower probability for the formation of a duplex containing mismatched bases. Thus, the higher the stringency, the greater the probability that two single-stranded polynucleotides, capable of forming a mismatched duplex, will remain single-stranded. Conversely, at lower stringency, the probability of formation of a mismatched duplex is increased. Similarly, at high stringency shorter polynucleotides will be less likely to stably hybridize or anneal to one another than will longer polynucleotides. Thus the ability of two single stranded polynucleotides to hybridize will therefore depend upon both their degree of complementarity and their overall length as well as the stringency of the hybridization reaction conditions. In general, longer polynucleotide hybrids have better duplex stability and thus require higher stringency conditions to prevent annealing or to decouple after annealing.

Methods of increasing the stringency of an assay or wash step include increasing the temperature, decreasing the salt concentration, certain modifications of pH, and/or the use of a destabilizing or denaturing agent including certain detergents, certain organic or inorganic solvents such as formamide, or certain modifications of pH, all of which tend to lower the Tₘ of polynucleotide duplexes. These and many other means of affecting Tₘ and thereby enhancing or disrupting specific interactions between polynucleotides are well-known in the art.

Typically, the stringency of the conditions used during hybridization and washing steps will be sufficiently high to wash away background polynucleotides (e.g., those that have a higher degree of sequence mismatch than desired) while allowing targets to remain substantially bound to the probes on the array.

A number of methods available in the art can be used to detect the bound targets. One preferred method is the detection of the bound polynucleotides via a detectable label, such as a fluorescent label, being attached to or incorporated as part of the target. The quantity and/or type of target specifically hybridized to an array, array bead, or microarray probe or spot is generally measured by, for example, previous fluorescent or radioactive labeling of the targets and the reading of the quantity of label present after hybridization on each probe, or by using other measuring methods of the quantity of probe-target hybridization for each probe, such as, for example, the measurement of micro-currents induced through the formation of a double strand probe-target electric capacitance, or the direct measurement of the molecular mass of the target fixed on each probe. Such methods of measurement are well-known in the art and any suitable method known in the art can be used in the practice of the present disclosure.

Removal of Hybridized Targets

To allow reuse of an array, the targets bound to the array are exposed to a degradative agent that chemically modifies at least some, most, or substantially all, of the nucleotide analogs present in the targets such that there is a reduction in the Tₘ of the hybridized probe-target duplexes. The Tₘ can be reduced anywhere from 2° C. to 60° C., allowing an array to be used 1 to 20 times, or more.

In preferred embodiments, DNA glycosylase is used to create an abasic site at anywhere from about 5% to about 100% of the nucleotide analogs present in a set of hybridized targets, or any number in between. As used herein, a “DNA glycosylase” is an enzyme that cleaves the N-glycosyl bond between a target base and deoxyribose, thereby releasing a free base and leaving an apurinic/apyrimidinic (AP) site in the polynucleotide strand. Many DNA glycosylases are present in nature and act specifically upon certain substrate bases. Examples of DNA glycosylases include, but are not limited to, uracil-DNA glycosylases (UDG/UNG) such as viral, bacterial, plant and human UNGs and Saccharomyces cerevisiae UNG1. Glycosylases also include akylbase-DNA glycosylases such as Escherichia coli TAG and alkA, S. cerevsiae 3-methyladenine DNA glycosylase gene (MAG), Schizosaccharomyces pombe MAG1, rodent/human N-methyl purine glycosylases (MPG) and Arabidopsis thaliana MPG. Also included are DNA glycosylases that remove oxidized pyrimidines (EndoIII-like), including but not limited to E. coli EndoIII (nth), S. cerevisiae NTG1, S. pombe NTH and bovine/human EndoIII homologue. Also included are Endo VIII and EndoIX glycosylases such as those from E. coli, mouse/bovine hydroxy-methyl-DNA glycosylase, and human formyluracil-DNA glycosylase. Other glycosylase include DNA glycosylases that remove oxidized purines such as E. coli 2,6-dihydroxy-5N-formamiodopyrimidine (Fapy) DNA glycosylase (FPG), S. cerevisiae 8-oxoguanosine DNA glycosylases 1 and 2 (OGG1 and OGG2), and Drosophila melanogaster S3, as well as pyrimidine-dimer DNA glycosylases from T4 bacteriophage, Neisseria mucosa and Micrococcus luteus, and 5-methyl-cytosine-DNA glycysolase.

As used herein, “uracil DNA glycosylase” (UDG) refers to any enzyme which catalyzes the hydrolysis of the N-glycosylic bond between the uracil and sugar, leaving an apyrimidinic site in uracil-containing single or double-stranded DNA. The result of treating targets comprising nucleotide analogs with a suitable DNA glycosylase is a reduction in the Tₘ of the hybridized duplexes of target and bound probe. The effect is comparable to creating mismatches between the strands in the hybridized duplex because the activity of the DNA glycosylase results in the complete removal of one of the bases in a base pair within the double-stranded hybrid. Thus, the target can be removed or stripped from the array using lower stringency conditions than otherwise would have been required.

Targets also can be further treated with an enzyme, such as a nuclease (e.g., an apurinic or apyrimidinic endonuclease), to further degrade the treated targets. Certain nuclease, for example, will nick a DNA strand on the 3′ or 5′ side of the lesion created by DNA glycosylase removal of a base. Such treatment further degrades the target and produces multiple smaller nucleic acid fragments from a single longer strand. As is well understood in the art, shorter polynucleotide duplexes typically have a significantly lower Tₘ than longer duplexes, and thus require much lower stringency conditions to be removed from the probes on the array. In any event, a greater degree of degradation can permit more efficient removal of the targets from the array after hybridization, thereby increasing the reusability of the array.

Removal of the targets after treatment with a DNA glycosylase and/or other degradative agent may be accomplished by heating and/or, by using chemical methods well known in the art for removing hybridized polynucleotides (e.g., exposing the treated targets to formamide, detergent, monovalent cation salt, or hydroxide).

In some embodiments, when sufficient degradation of the nucleotide analogs within the targets has been achieved, the enzymatic reaction is terminated to prevent degradation of subsequent targets hybridized to the array. “Terminating” as used herein means causing a treatment to stop. The term includes termination that is both permanent and conditional. For example, if the treatment is enzymatic, a permanent termination would be achieved by heat and/or chemical denaturation; whereas a conditional termination would be achieved, for example, by using a temperature outside the enzyme's active range, but not sufficient to denature or otherwise damage the enzyme. Both types of termination are intended to fall within the scope of this term. A DNA glycosylase and/or a nuclease such as uracil DNA glycosylase may be rendered inactive, for example, through heat, protease treatment, inhibitor, antibody, or dilution.

Once sufficient degradation of the targets has been achieved, the array can then be stripped and, if desired, reused for subsequent assays. As disclosed herein, the degradation and stripping conditions can remove all, substantially all, most, or at least some portion of the targets bound to the array. For example, using the disclosed methods, most (e.g., 60-100%, 70-100%, 80-100%, 90-100%, 95-100% or 99-100%) of the hybridized targets can be removed from the array. The conditions are also preferably mild enough so as to prevent substantial damage to the array, and the probes bound to the array. Methods of determining appropriate stripping conditions and implementing those conditions to achieve optimal results using the methods and compositions disclosed herein are well within the skills of those in the art.

Compositions and Kits

Disclosed are compositions comprising an array of probes hybridized to targets comprising nucleotide analogs that confer susceptibility to a degradative enzyme, and a degradative agent. In certain embodiments the array will be a spatially-addressable array or microarray. In other embodiments, the DNA glycosylase and/or other degradation agent are in contact with the array, preferably via a suitable buffer solution, e.g. one that permits enzymatic action by the glycosylase or other degradation agent.

The present disclosure provides kits for incorporating nucleotide analogs into targets and for stripping an array comprising a labeling buffer, polymerase, nucleotides, nucleotide analogs, primers, a stripping buffer and a DNA glycosylase, a nuclease and/or other degradation agent. A stripping buffer is a solution optimized to remove targets from an array while retaining enough of the original features of the array as to permit its reuse in subsequent assays of different sets of targets.

Typical stripping buffers are ones which contain an organic solvent, most commonly formamide. The organic solvent is present in the stripping solution at a concentration anywhere from 0% to 90% v/v, and preferably between 30% to 70% v/v. Alternately or additionally, a stripping buffer may contain a detergent or other denaturing agent. The concentration of such denaturation agents can vary anywhere from 0.0001% to 20% v/v or w/v, and is preferably between 0.001% and 10% v/v or w/v. A stripping buffer may also contain a salt, preferably a salt comprising a monovalent cation. The salt concentration can vary anywhere from 0.01 nM to 4M, and preferably between 500 nM and 1M. A stripping buffer may contain one or more agents to make the buffer basic or acidic. For example, the agent may lower the pH to anywhere from 1 to 7, i.e., increase the H⁺ concentration. Alternatively, the agent may raise the pH to a value between 7 and 14, i.e., increases the OH⁻ concentration.

In certain embodiments, the kit can contain a DNA glycosylase, a nuclease and/or other degradation agents capable of chemically-modifying a nucleotide analog. The DNA glycosylase, nuclease and other degradation agent can be provided in any suitable form that retains its enzymatic activity with regard to chemically-modifying nucleotide analogs or which permits enzymatic activity to be restored to the agent before exposure to a nucleotide analog containing target. For example, the glycosylase, nuclease and/or other agent can be provided as a lyophilized powder or in an appropriate buffer solution.

In further embodiments, the kit can include nucleotide analogs. Nucleotide analogs can be provided in any form that retains their suitability for use in nucleic acid incorporation, or which allows them to be readily modified for such use.

### EXAMPLE 1

### Synthesis and Stability of dU-Containing DNA

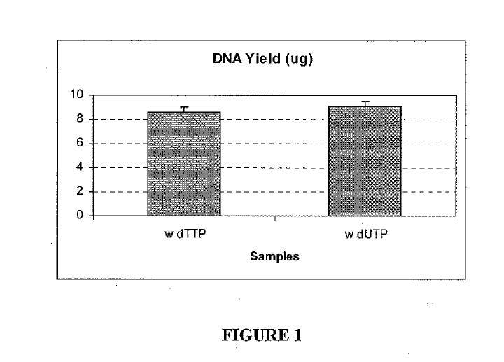

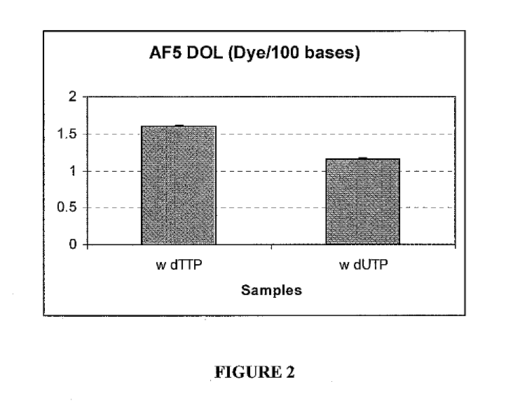

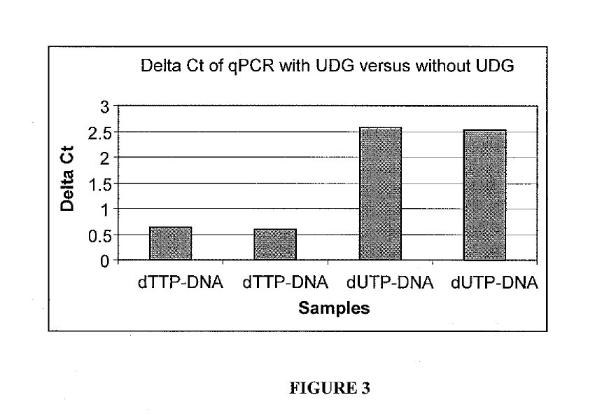

dUTP-containing DNA was synthesized and purified using the BioPrime™ Total Genomic Labeling System (Catalog Number: 18097-010, 18097-011, 18097-012) protocols. The DNA yield for the synthesis is shown in FIG. 1. Dye incorporation is shown in FIG. 2. Quantitative PCR was used to verify that dUTP was effectively incorporated into the synthesized DNA. The results of that assay are shown in FIG. 3. The difference in delta Ct between dU-DNA samples treated or not treated with UDG confirm that dUTP was indeed incorporated into the DNA.

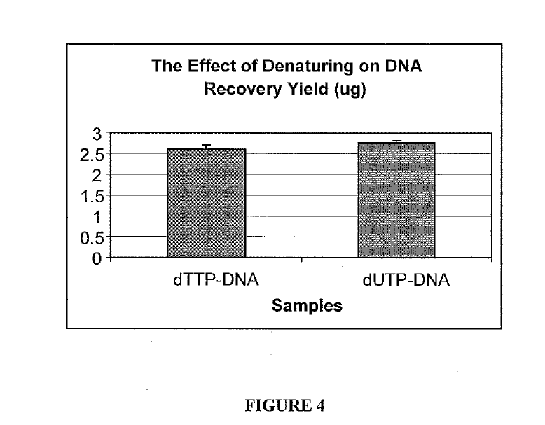

To test the stability of the dUTP-containing oligonucleotides under denaturing conditions, 20 μl of dUTP-containing oligonucleotides were added in the same volume of 100 mM Tris-HCl (pH 8.0). Reaction tubes were incubated at 95° C. for 10 minutes, cooled to room temperature (˜25° C.), and then purified as above. DNA recovery is shown in FIG. 4.

### EXAMPLE 2

### Arrays of Probes Hybridized to dU-Containing DNA Targets

To analyze the hybridization efficiency of dU-containing oligonucleotides onto an array, dT-DNA, dU-DNA, and negative control samples were hybridized on a Macrogen™ CGH array.

Samples were mixed with Cot-1 DNA, sodium acetate, and ethanol as follows:

| | |
|---|---|
| Test DNA (AF3 labeled) | 80 μl |
| Reference DNA (AF5 labeled) | 80 μl |
| Cot-1 DNA (1 mg/ml) | 100 μl |
| Sodium Acetate | 30 μl |
| 100% Ethanol | 600 μl |

The sample mixture was put at −20° C. for 1 h in the dark. Then it was centrifuged at 13000 rpm for 20 min at 4° C. Supernatant was removed. 500 μl of 70% ethanol was added. The sample was centrifuged at 13000 rpm for 10 min at 4° C. Next, the supernatant was removed and the pellets were allowed to air-dry for 10 min. The hybridization solution was prepared by adding 50 μl of Solution C (according to the manufacture's protocol) and 5 μl of Yeast tRNA (50 mg/ml) to the dried sample. The hybridization solution was allowed to stand for >30 min (up to 24 h) in the dark. The tube was occasionally tapped to dissolve the DNA.

Prehybridization solution was prepared by mixing 30 μl of Solution C and 10 μl Salmon Sperm DNA (10 mg/ml). The prehybridization solution was vortexed and then spun down briefly, and incubated at 70° C. for 10 min and then 0° C. 5 min. 40 μl of prehybridization solution was applied onto the spotting area of the array, then the glass slide was covered with cover glass with out any air bubbles. Array slides were incubated for 30 min at room temperature in the dark. Slip covers were removed and arrays were inserted immediately in fresh distilled water. Slides were slightly agitated by moving up and down for 10 sec. The arrays were transferred to new container of fresh water and agitated for 10 sec. The arrays were next transferred into a container of 2-propanol and agitated for 10 sec. Finally, the arrays were dried by centrifugation at 500 rpm.

For hybridization, hybridization solution was incubated at 70° C. for 15 min and then at 37° C. for 60 min. Next, the hybridization solution was added to the array. The array was incubated at 37° C. 42 h using the Maui Hybridization System (Biomicro System, Model no. 02-A002-02). The coverslip was removed from the array and the array was washed as set forth below and using the solutions set forth in Table 1:
1. In Washing Solution 1, agitate 40 times and then stand for 15 min at 46° C.
2. In Washing Solution 2, agitate 40 times and then stand for 30 min at 46° C.
3. In Washing Solution 3, agitate 40 times and then stand for 15 min at RT.
4. In Washing Solution 4, agitate 40 times and then stand for 5 min at RT.
5. In 70% Ethanol, agitate 20 times.
6. In 85% Ethanol, agitate 20 times.
7. In 100% Ethanol, agitate 20 times.

TABLE 1

| Washing | 46° C. | 46° C. | RT | RT | RT | RT | RT |
|---|---|---|---|---|---|---|---|
| Components | 1 | 2 | 3 | 4 | 70% | 85% | 100% |
| 20XSSC | 20 ml | 20 ml | | 25 ml | | | |
| Distill Water | 80 ml | 180 ml | 100 ml | 225 ml | 60 ml | 30 ml | |
| Formamide | 100 ml | | | | | | |
| 2×Phosphate Buffer | | | 100 ml | | | | |
| 20% SDS | | 1 ml | | | | | |
| Nonidet P40 | | | 200 μl | | | | |
| Ethanol | | | | | 140 ml | 170 ml | 250 ml |
| Total Volume | 200 ml | 200 ml | 200 ml | 250 ml | 200 ml | 200 ml | 250 ml |
| Agitate | 40 | 40 | 40 | 40 | 20 | 20 | 20 |
| Time (min) | 15 | 30 | 15 | 5 | | | |

Arrays were air dried and scanned on an Axon™ scanner GenePix 4000B at 800 PMT for both 532 nm and 635 nm channels. Data was processed using GenePix Pro 6.0. The average signal was determined as shown below in Table 2:

TABLE 2

| | AF5 |
|---|---|
| Negative Control | 2 |
| dU-DNA | 734 |
| dT-DNA | 4730 |

### EXAMPLE 3

### UDG Treatment of Arrays

Strip array with 50% formamide. The array with the hybridized dUTP-DNA was incubated in 50% formamide and 10 mM Tris-HCl pH 7.5 for 40 minutes at 65° C., and then washed with the same solution at room temperature (˜25° C.). The array was subsequently washed with 70%, 85%, and then 100% ethanol.

Strip array with UDG. The array was treated with UDG by incubating it with Heat-Labile UDG (1 unit/100 μl) for 2 hours at 37° C., and then incubated at 62° C. for 10 minutes. Next, the array was incubated at 55° C. for 30 minutes in 50% formamide and 10 mM Tris-HCl pH 7.5, and then washed in the same solution at room temperature (˜25° C.). The array was finally washed with 70%, 85%, and then 100% ethanol.

The average signal is shown below in Table 3:

TABLE 3

| | Negative | dUTP- | dTTP- |
|---|---|---|---|
| Average Signal | Control | DNA | DNA |
| Formamide | 1 | 134 | 137 |
| UDG treatment | 3 | 31 | 77 |

After stripping with 50% formamide, the average signal from dU-DNA was almost the same as that from dT-DNA. UDG treatment decreased average signal from dU-DNA by about 77%, while the average signal from dT-DNA decreased by only about 44%.

### EXAMPLE 4

### UDG Treatment and Reuse of Arrays

Preparation of labeled gDNA for hybridization. Male and female human genomic DNA was digested with restriction enzymes, Alu I and Rsa I. dUTP and AF-dCTP were incorporated into genomic DNA using the labeling protocol as described in Example 1. 50 μl of AF3 labeled human male gDNA and 50 ul of AF5 labeled human female gDNA were mixed. The mixed samples were put in a speed-vac to reduce the volume to less than 39 μl. Nuclease-free water was added to 39 μl.

Next, the following components were added to nuclease-free tubes in the order indicated:

| | Volume (μl) |
|---|---|
| Components | per hybridization |
| AF3 and AF5 labeled gDNA | 39 |
| Cot-1 DNA (1.0 mg/ml) | 5 |
| Agilent 10x Blocking Agent | 11 |
| Agilent 2x Hybridization Buffer | 55 |
| Final hybridization sample volume | 110 |

Samples were incubated at 95° C. for 3 minutes. Samples were immediately transferred to 37° C. and incubated at 37° C. for 30 minutes. Tubes were centrifuged for 1 minute at 10,000×g to collect the samples at the bottom of the tubes.

Hybridization of dU-containing gDNA onto array. Clean Agilent 4×44K gasket slides were loaded into an Agilent SureHyb chamber base with the gasket label facing up and aligned with the rectangular section of the chamber base. Slowly, 100 μl of hybridization sample mixture was dispensed into the gasket wells. Agilent 4×44K CGH microarray “active sides” were placed down onto the gasket slides so the numeric barcode side was facing up. The sandwich-pair was properly aligned and the chamber cover was placed onto the sandwiched slide. The clamp assembly was then slid onto both pieces and hand-tightened onto the chamber. The assembled chamber was vertically rotated to wet the slide and assess the mobility of the bubbles. For hybridization, the slides were then incubated at 65° C. for 24 hours at 20 rpm.

After hybridization, the chamber was disassembled and slides were washed according to the following procedure:

| | Dish | Wash Buffer | Temperature | Time |
|---|---|---|---|---|
| Disassembly | 1 | Wash Buffer 1 | Room temp. | |
| 1^{st} wash | 2 | Wash Buffer 1 | Room temp. | 5 min |
| 2^{nd} wash | 3 | Wash Buffer 2 | 37° C. | 1 min |
| Acetonitrile | 4 | Acetonitrile | Room temp. | 1 min |
| 3^{rd} wash | 5 | Stabilization and | Room temp. | 30 sec. |
| | | Drying Solution | | |

Analysis of arrays with probes hybridized to dU-containing gDNA. Arrays were scanned on an Axon™ scanner GenePix 4000B at 600 PMT using both channels. Pixel size was 5 μm. Data was processed using GenePix Pro 6.0.

UDG treatment of arrays. Slides were incubated in Acetonitrile for 2 minutes at room temperature. They were then washed in 5 mM Tris-HCl pH 7.5 for 5 min at room temperature. This wash was repeated twice. Slides were incubated with heat-labile UDG (10 uint/100 ul) in 70 mM Tris-HCl pH 8.0, 50 mM NaCl, 1 mM EDTA for 2 hours at 37° C. Then incubated at 50° C. for 30 minutes. Slides were washed in 5 mM Tris-HCl pH 7.5 at 35° C. for 15 minutes. This wash was repeated twice.

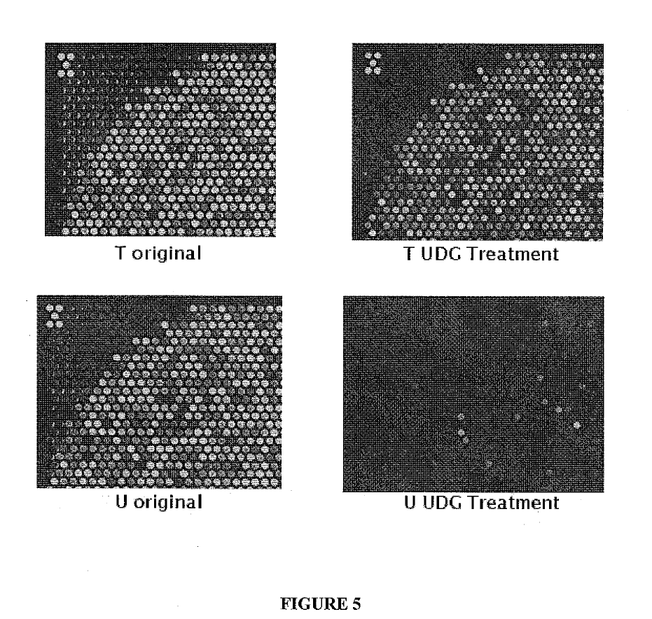

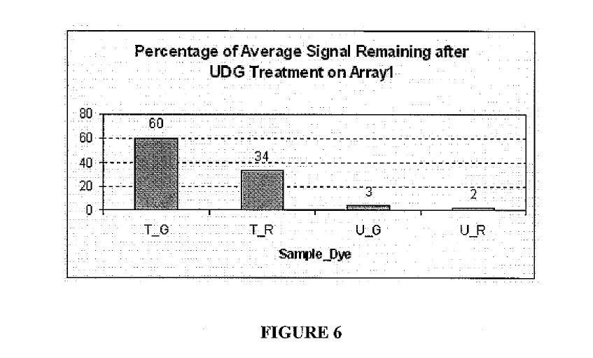

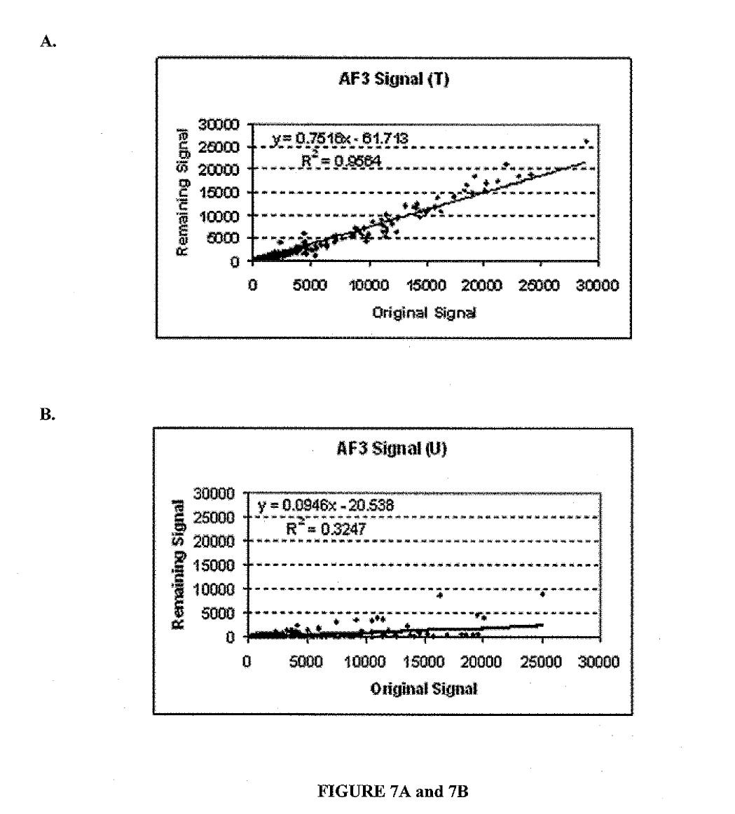

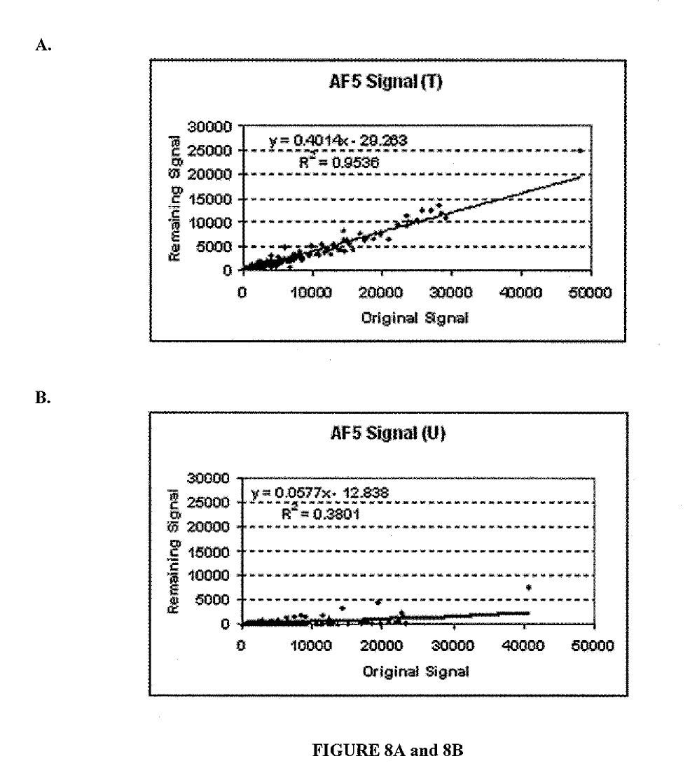

Analysis of arrays after UDG treatment. Arrays were scanned on an Axon™ scanner GenePix 4000B at 600 PMT using both channels and imaged using GenePix Pro 6.0 (pixel size 5 μm). See FIG. 5. Signals from dUTP containing samples collectively decreased 17-20 fold after UDG treatment. See FIG. 6. Signal decrease after UDG treatment for individual features is shown in FIGS. 7 and 8.

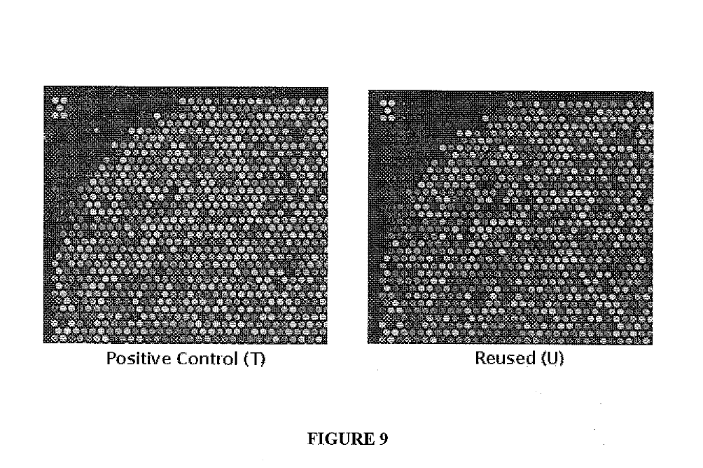

Reuse of arrays after UDG treatment. UDG treated arrays were hybridized with AF3 labeled human male gDNA and AF5 labeled human female gDNA, and analyzed as above. See FIG. 9. Log2 ratios for chromosome 1, chromosome X, and chromosome Y features for the reused array indicated the targets rehybridized with the expected specificity.

### EXAMPLE 5

### UDG+APE 1 Treatment and Reuse of Arrays

Sample preparation, labeling, and hybridization was performed as in Example 4, with the substitution of the following alternative post-hybridization wash procedure:

| | Dish | Wash Buffer | Temperature | Time |
|---|---|---|---|---|
| Disassembly | 1 | Wash Buffer 1 | Room temp. | |
| 1st wash | 2 | Wash Buffer 1 | Room temp. | 5 min |
| 2^{nd} wash | 3 | Wash Buffer 2 | 37° C. | 1 min |

UDG+APE 1 treatment of arrays. Slides were incubated with heat-labile UDG (10 uint/100 ul) in 50 mM potassium acetate, 20 mM Tris-acetate, 10 mM magnesium acetate, 1 mM DTT for 2 hours at 37° C. and then incubated at 55° C. for 30 minutes. Slides were washed in 5 mM Tris-HCl pH 7.5, 0.05% SDS, 50% formamide at room temperature for 15 minutes. The wash was repeated 3 times. Slides were rinsed in 5 mM Tris-HCl pH 7.5, 0.05% SDS at room temperature. 600 ml of 100 mM potassium phosphate buffer (pH6.6, filtered) was added to a 2 liter beaker. The beaker was microwaved until the buffer reached 65° C. Once the buffer was warmed, it was transferred to a hotplate. A magnetic stir bar and thermometer were added. The rack containing the slides was then added. The beaker was covered with aluminum foil and an insulating cover, and the buffer was stirred at medium-low speed. Buffer was again heated until it reached 100° C. and there was a steady stream of bubbles (approximately 10 minutes). Slides were left in hot buffer for 5 minutes during which time the buffer reached a rolling boil. Slides were removed from the beaker and excess liquid was blotted dry. Quickly, the slide rack and slides were plunged into a glass dish containing 250 ml of 100 mM potassium phosphate buffer (pH6.6, filtered). Slides were allowed to cool for 2 minutes. Slowly, the slides were removed from the buffer and then allowed to dry.

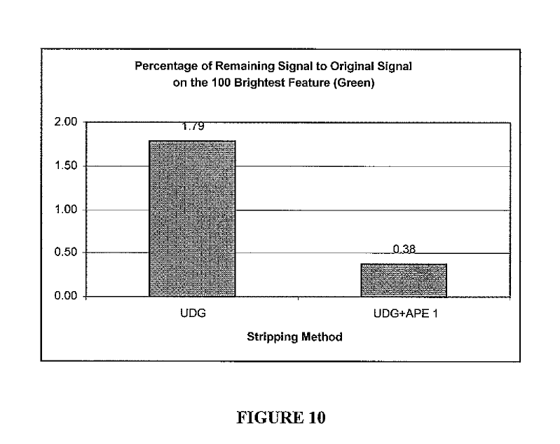

Analysis of arrays after UDG+APE1 treatment. Arrays were scanned on an Axon™ scanner GenePix 4000B at 600 PMT using both channels and imaged using GenePix Pro 6.0 (pixel size 5 μm). The average remaining signal for the 100 brightest green channel features for UDG+APE 1 treated arrays was more than 4.5× reduced compared to UDG treatment alone. See FIG. 10.

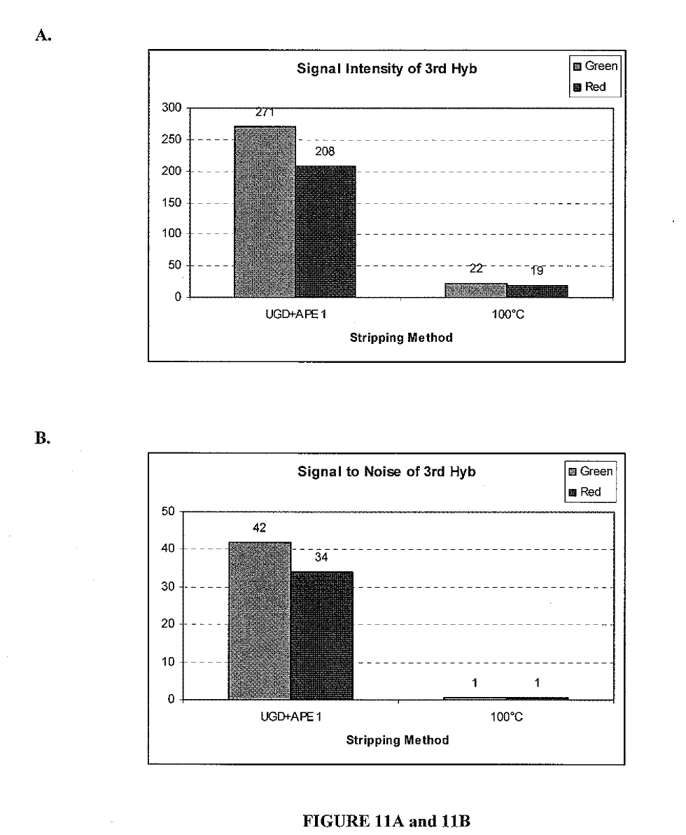

Reuse of arrays after UDG+APE1 treatment. Three cycles of hybridization, UDG+APE1 treatment, stripping and rehybridized were performed as above. A 100° C. stripping control was used to establish baseline signal. See FIGS. 11A and 11B.

Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

What is claimed is:

1. A method for rendering a nuclide acid array suitable for repeated use, comprising:
a. providing targets comprising a nucleotide analog that confers susceptibility to a degradative agent;
b. providing an array having probes attached thereto;
c. performing hybridization of said targets to said probes; and
d. incubating said array with said degradative agent, whereby most of said targets are removed from said array.

2. A method for removing targets hybridized to probes of a nucleic acid array, wherein said targets comprise a nucleotide analog that is susceptible to modification by a degradative agent, comprising:
incubating said array with said degradative agent, thereby removing most of said targets from said array.

3. The method of claim 1 or 2, wherein said degradative agent chemically modifies one or more of said nucleotide analogs.

4. The method of claim 3, wherein said chemical modification renders one or more said nucleotide analogs abasic.

5. The method of claim 3, wherein said chemical modification reduces the thermal stability (Tₘ) of said hybridized target and probe.

6. The method of claim 1 or 2, wherein said probes are immobilized on a solid support.

7. The method of claim 6, wherein said probes are covalently bound to said solid support.

8. The method of claim 1 or 2, wherein said probes are DNA.

9. The method of claim 1 or 2, wherein said targets are DNA.

10. The method of claim 1 or 2, wherein said targets are detectably labeled.

11. The method of claim 10, wherein said label is a fluorescent label.

12. The method of claim 10 wherein said label is a biotin label.

13. The method of claim 1 or 2, wherein said targets are single-stranded.

14. The method of claim 1 or 2, wherein said array is a microarray.

15. The method of claim 1 or 2, wherein said array is a spatially-addressable array.

16. The method of claim 1 or 2, wherein said array is a bead-based array.

17. The method of claim 1 or 2, wherein said nucleotide analog is a base-modified nucleotide.

18. The method of claim 17, wherein said nucleotide analog is deoxyuridine monophosphate (dUMP) or deoxyuridine triphosphate (dUTP).

19. The method of claim 1 or 2, wherein said degradative agent is a glycosylase.

20. The method of claim 19, wherein said glycosylase is uracil DNA glycosylase.

21. The method of claim 1 or 2, wherein said nucleotide analogs are not present in any or most of the probes immobilized on the array.

22. The method of claim 3, wherein said chemically modified targets are removed from the immobilized probes through heat, protease treatment, or dilution.

23. The method of claim 22, wherein said protease is an apurinic or apyrimidinic endonuclease.

24. The method of claim 1 or 2, further comprising hybridizing a different set of targets to the probes.

25. The method of claim 24 wherein said different set of targets comprises one more of said nucleotide analogs.

26. The method of claim 1 or 2, wherein 80-100% of the hybridized targets are removed.

27. The method of claim 1 or 2, wherein 90-100% of the hybridized targets are removed.

28. The method of claim 1 or 2, wherein 99-100% of the hybridized targets are removed.

29. A composition comprising:
a. a nucleic acid array comprising probes hybridized to targets comprising a nucleotide analog that is susceptible to modification by a degradative agent; and
b. said degradative agent.

30. The composition of claim 29, wherein said probes are immobilized on a solid support.

31. The composition of claim 30, wherein said probes are covalently bound on a solid support.

32. The composition of claim 29, wherein said probes are DNA.

33. The composition of claim 29, wherein said targets are detectably labeled.

34. The composition of claim 33, wherein said label is a fluorescent label.

35. The composition of claim 33, wherein said label is a biotin label.

36. The composition of claim 29, wherein said targets are DNA.

37. The composition of claim 29, wherein said targets are single-stranded.

38. The composition of claim 29, wherein said array is a microarray.

39. The composition of claim 29, wherein said array is a spatially-addressable array.

40. The composition of claim 29, wherein said nucleotide analog is a base-modified nucleotide.

41. The composition of claim 40, wherein said nucleotide analog is deoxyuridine monophosphate (dUMP) or deoxyuridine triphosphate (dUTP).

42. The composition of claim 29, wherein said degradative agent is a glycosylase

43. The composition of claim 42, wherein said glycosylase is uracil DNA glycosylase.

44. The composition of claim 29, wherein said nucleotide analogs are not present in any or most of the probes immobilized on the array.

45. The composition of claim 29, further comprising a nuclease or stripping buffer.

46. A kit for removing targets hybridized to an array, comprising:
a. one or more stripping buffers; and
b. one or more glycosylases.

47. The kit of claim 46, wherein said stripping buffer comprises at least 5% (w/v or v/v) formamide, at least 0.0001% (v/v or w/v) detergent, at least 0.01 nM monovalent cation salt, at least 0.2 nM OH⁻, or any combination thereof.

48. The kit of claim 46, further comprising a nuclease.

49. The kit of claim 46, further comprising nucleotide analogs.

50. The kit of claim 46, further comprising a nucleic acid polymerase.

51. The kit of claim 48, wherein said nuclease is an apurinic/apyrimidinic endonuclease.
